(19) United States
(12) Patent Application Publication
(10) Pub. No.: US 2006/0037100 A1
(43) Pub. Date: Feb. 16, 2006

(54) Fungal resistant transgenic pepper plants and their production method

(76) Inventors: Young Soon Kim, Nam-Gu (KR); Moon Kyung Ko, Suncheon-Shi (KR); Hyo Hyoun Seo, Kwangsan-Gu (KR); Jung Hyun Cho, Buk-Gu (KR); Pill-Soon Song, Kwangsan-Gu (KR)

Correspondence Address: KENYON & KENYON, 1500 K STREET NW, SUITE 700, WASHINGTON, DC, 20005, US

(21) Appl. No.: 10/916,419

(22) Filed: Aug. 12, 2004

(51) Int. Cl. C12N 15/82; A01H 1/00; C12N 15/87

(52) U.S. Cl. 800/279

(57) ABSTRACT

A stable pepper transformation was established using Agrobacterium mediated method. Pepper plants were transformed with PepEST or PepDef gene, where the expression of the nucleic acid sequence in the plant resulted in increased resistance to fungal infection as compared to the wild type plant. Provided are agricultural products including seeds produced by the transgenic plants. Also provided are vectors and host cells containing the nucleic acids coding PepEST and PepDef, respectively.

### FIELD OF THE INVENTION

The present invention relates to transgenic pepper plants resistant to anthracnose fungus and a method for producing the said transgenic pepper plants. More specifically, the present invention relates to novel distinctive pepper transgenic lines carrying PepEST or PepDef genes as well as methods for their production.

### BACKGROUND OF THE INVENTION

Capsicum annuum L. (pepper) is an important vegetable crop characterized by high earning crop around the world. However, all commercial varieties are susceptible to anthracnose fungus, which may result in 10-15% losses in annual yield. So, current goal of pepper biotechnology is to increase pepper's resistance to anthracnose fungus. To date, improvement of pepper has been restricted to conventional breeding since the transformation of pepper was not routinely applicable to introduce valuable genes into the genome.

A transformation system of plants requires tissue cultures competent for efficient plant regeneration as well as an effective method of gene delivery. In pepper, tissue culture techniques have been used to produce somatic embryos and haploid plants. And its ‘in vitro’ regeneration has been reported by numerous laboratories. Despite of the tissue cultural advantage of this species, pepper has been known as a recalcitrant plant to be genetically transformed. There have been no reports on genetically stable transgenic peppers although a few attempts have been made to transform pepper plants in recent years. Here, we established an efficient transformation method for pepper using Agrobacterium tumefaciens and also demonstrated stable inheritance of the transgenes in transgenic peppers.

Plant transformation involves the transfer of desired genes into the plant genome and then the regeneration of a whole plant from transformed cells. To transfer genes into plants, Agrobacterium is widely used in many plant species. Agrobacterium infection and gene transfer normally occur at the site of a wound in the plant. In the case of pepper, Agrobacterium infection causes severe accumulation of phenolic compounds at the infection sites, and further growth of the tissues is thus arrested. So, the protocol for tissue culture method was designed to circumvent growth retardation after Agrobacterium infection. Transformants were then selected by their ability to divide and grow in tissue culture medium with antibiotics. Here, we report a reproducible method for the stable transformation of pepper plants using Agrobacterium.

With the advent of genetic engineering, introducing disease resistant genes such as PepEST (U.S. Pat. No. 6,018,038) and PepDef (U.S. Pat. No. 6,300,489) have led to the development of transgenic peppers. PepEST gene encoding a member of esterase was isolated from the ripe pepper fruits that showed incompatible interaction with anthracnose fungus. The PepEST protein plays dual roles in the plant-pathogen interaction, namely direct inhibition of fungal infection by arresting appressorium formation and by activating the disease-resistant signaling pathway. On the other hand, many plant defensins can inhibit the growth of a broad range of fungi at micromolar concentrations but are nontoxic to both mammalian and plant cells. Plant defensins are structurally related to insect defensins such as drosomycin, which is an antifungal peptide found in Drosophila melanogaster. The PepDef protein that belongs to a defensin family is small cysteine-rich peptides with antimicrobial activity. Thus, PepEST and PepDef genes, respectively, were introduced into pepper plants to control anthracnose fungal disease. The present invention relates to new distinctive pepper transgenic lines carrying PepEST or PepDef genes and the method for their production as well.

### SUMMARY OF THE INVENTION

According to the invention, new transgenic pepper lines, designated as PepEST transgenic pepper (PepEST-TP) and PepDef transgenic pepper (PepDef-TP) respectively, are provided. This invention thus relates to plants of transgenic pepper lines, to seeds of transgenic pepper lines and to methods for producing the transgenic pepper plants mediated by Agrobacterium. This invention also relates to establishment for producing other transgenic pepper lines derived from transgenic PepEST-TP and PepDef-TP. This invention further relates to hybrid the plants produced by crossing the transgenic PepEST-TP and/or PepDef-TP with other pepper lines.

### BRIEF DESCRIPTION OF THE DRAWINGS

FIG. 1 shows growth and regeneration of transgenic pepper explants. The sensitivity of untransformed wild type (WT) and transformed (T) hypocotyl explants to hygromycin B was examined by using shoot induction medium containing 0, 5, 10, 20, 50 mg/l of hygromycin B (A). Fresh weight (mg) of hypocotyl explants growing on the medium containing hygromycin B (B).

FIG. 2 shows the integration and expression of GUS gene in transgenic pepper plants. Genomic DNA was digested with enzymes that produced a single cut in the T-DNA. ³²P-labeled GUS probe was used for hybridization. The result indicates that a single copy of T-DNA of pCAMBIA1301 was integrated in the respective transgenic lines (A). Nine independent transgenic pepper lines were analyzed for the expression of GUS following Agrobacterium infection carrying pCAMBIA 1301 or 1304. The result indicates that GUS gene was expressed in nine transgenic lines tested (B).

FIG. 3 shows GUS specific activities conducted in situ (A-a) or in extracts (A-b) of various tissues from a transgenic pepper that contain the GUS reporter gene. T₁ transgenic plants were screened from the progenies of the T₀ plant by using hygromycin B resistance. GUS staining was also used to examine the expression of GUS gene in transformed progenies. Out of the 21 harvested seeds of transgenic pepper (No. 2), 15 seedlings gave rise to transgenic progenies showing resistance to hygromycin B (20 mg/l). All of the resistant plants were GUS positive (B).

FIG. 4 shows the integration and expression of PepEST gene in transgenic pepper plants (PepEST-TP). Genomic DNA was digested with HindIII restriction enzyme that produced a single cut in the T-DNA. ³²P-labeled PepEST probe was used for hybridization. The result indicates that 1 or 2 copies of T-DNA was integrated into the genome of respective transgenic line (A-a). Seven transgenic lines were analyzed for the expression of PepEST gene by Northern hybridization. (A-b). In addition, T₁ seedlings showed the stable inheritance of the T-DNA (B). Southern blotting of transgenic progenies derived from a transgenic plant (No. 21) revealed the same integration pattern of PepEST gene shown in their parental plant, and HPT as well.

FIG. 5 represents the SDS-PAGE of soluble proteins extracted from transgenic plants (PepEST-TP) (A-a) and Western analysis of same fractions as in (a) showing the PepEST protein band (A-b). The amount of PepEST protein was measured in the soluble proteins using ELISA method (B).

FIG. 6 shows the resistance of the unripe pepper fruits from transgenic plants carrying PepEST or PepDef. Inoculated fruits were photographed at 9 days after infection of anthracnose fungus. As control, the unripe fruits of wild type plant were used.

### DETAILED DESCRIPTION OF THE INVENTION

Followings are detailed description of embodiments of the present invention.

Plant Material

Pepper seeds were surface sterilized for 5 min in 0.2% sodium hypochlorite followed by several rinses with sterile distilled water and then germinated on Murashige and Skoog (MS) medium in the dark. After 7 days of incubation, seedlings were exposed to light for 6 hr. Then, cotyledons and hypocotyls were excised for inoculation of Agrobacterium.

Construction of the Transformation Vector

A full length cDNAs of PepDef gene (SEQ ID NO: 1) and PepEST gene (SEQ ID NO: 2) were isolated from pepper. The PepEST cDNA was amaplified by PCR using a forward primer sequence with a BamHI restriction site (5′-ggatccaaaatggctagccaaagttttgttcc-3′: SEQ ID NO: 3) and a reverse primer sequence (5′-aatttgtagtagcacatatgaa-3′: SEQ ID NO: 4). This fragment was subcloned into BamHI- and Smal-digested pBI121. Then, the expression cassette was restricted with HindIII and EcoRI and ligated into cloning sites of pCAMBIA 1300 and named as pCAM-EST. To clone PepDef cDNA, primers used were (5′-gggtctagaaaaatggctggcttttccaaagtg-3′: SEQ ID NO: 5) for the forward with XbaI and (5′-ctcggatcctaattaagcacagggcttcgt-3′: SEQ ID NO: 6) for the reverse with BamHI. Then, the PCR product was cloned into pCAMBIA1300 and named as pCAM-Def. Finally, the plasmid DNA was mobilized into A. tumefaciens GV3101, respectively.

For plant transformation, Agrobacterium strain GV3101 carrying the binary vectors were used. Agrobacteria were cultured to log phase in YEP medium at 28° C. The bacteria were resuspended and agitated for 4 hr in MS liquid medium containing 20 μM acetosyringon to induce the virulence.

Pepper Transformation

Pepper explants excised from the cotyledon were incubated on CIM medium (MS medium supplemented with 0.5 mg/l IAA and 0.2 mg/Zeatin) prior to inoculation with Agrobacterium. Following 48 hr incubation, the explants were submerged in the Agroacterium suspension for 5 min, blot-dried and co-cultured for 48 hr at 28° C. in the dark on CIM medium. Infected explants were transferred for selection to CIM medium with 500 mg/l cefotaxime and 20 mg/l hygromycin B for 2 weeks. Thereafter on every 2 weeks, the explants were subcultured onto SIM medium (MS medium supplemented with 0.2 mg/l IAA and 1 mg/Zeatin) containing both antibiotics to induce shoot regeneration. The shoots regenerated from calli were rooted on the MS medium containing 10 mg/l hygromycin B. The established plantlets were acclimated in a greenhouse for further analysis.

Inheritance Analysis of Transgenic Progenies

The transgenic pepper lines were maintained in greenhouse and self-fertilized to generate the seeds. The transgenic progenies were screened by hygromycin resistance that was provided by the hpt gene. Seeds of T₀ transgenic pepper were surface sterilized and placed on a half strength MS medium containing 20 mg/l hygromycin B for 7 days for germination. Finally, healthy green plants were counted and transferred to soil.

Screening of Transgenic Pepper by PCR

Polymerase chain reaction (PCR) was performed with the genomic DNA from putative transgenic plants and their progenies to examine the presence of transgenes. Sets of specific primers were used to amplify GUS, PepEST, and PepDef, respectively. The primer set consists of (i) forward primer designed based on the sequence of CaMV35S promoter, corresponding to nucleotide positions 847-874 and (ii) reverse primer described above for each gene. The PCR conditions were 5 min at 94° C., then 35 cycles of 94° C. for 30 sec and 30 sec for annealing at 60° C. with 1 min extension period at 72° C. The amplified fragments were separated on 1% agarose gels.

Southern Analysis

Genomic DNA from selected transgenic pepper plants was used for Southern hybridization. Ten μg of genomic DNA was digested with 50 units of Hind III or EcoRI for overnight. DNA gel blotting was performed and then prehybridization was carried out at 65° C. for 2 hr, followed by hybridization at 65° C. overnight with the [α-³²P] dCTP-labeled CDNA probe in the prehybridization solution. Radiolabeled probe was prepared by using a random primer-labeling kit. Then, the blots were washed once in 2×SSC, 0.1% SDS for 10 min at 65° C., and once in 0.1×SSC, 0.1% SDS. The blots were exposed to X-ray film.

Northern Analysis

Total RNAs were extracted from independent transgenic peppers by the RNeasy Plant Kit (QIAGEN) according to the manufacturer's instructions and stored at −80° C. RNA gel blotting was performed and prehybridization was carried out at 65° C. for 2 hr, followed by hybridization at 65° C. overnight with the [α-³²P] dCTP-labeled cDNA probe in the prehybridization solution. The blots were washed once in 2×SSC, 0.1% SDS for 10 min at 65° C., and once in 0.1×SSC, 0.1% SDS. The blots were exposed to X-ray film. Radiolabeled probe was prepared by using a random primer-labeling kit.

GUS Enzymatic Assay

GUS histochemical staining of transgenic plants was performed as described by Jefferson et al (1987) in a solution of 50 mM NaPO₄ (pH 7.0), 10 mM EDTA, 0.5 mM K₃[Fe(CN)₆], 0.5 mM K₄[Fe(CN)₆], 0.1% sarcosyl, 0.1% β-mercaptoethanol, 0.1% Triton X-100, 1 mg/ml X-gluc (5-bromo-4-chloro-3-indolyl-β-D-glucuronic acid) at 37° C. overnight. GUS fluorogenic assays of tissue samples from various organs were performed as described by Jefferson et al (1987). Extracts were assayed for GUS activity and protein concentrations were determined by Bradford assay (Bio-Rad). Fluorescence at time intervals was measured with excitation at 320-390 nm and emission at 415-650 nm by using a TD-700 fluorometer (Turner Designs, USA) and the slope was determined. The specific activity of the GUS enzyme was calculated as pmol 4-methyl umbelliferone (MU) min/mg total protein. GUS activity was estimated from the average of three replicate assays.

SDS-PAGE and Western Blotting

Protein samples were extracted from leaves or fruits directly in 2× loading buffer and separated by SDS-PAGE. Protein concentrations were determined by Bradford assay (Bio-Rad). Proteins were transferred to PVDF membranes (Bio-Rad) and blocked in 5% skim milk powder in TBS (10 mM Tris (pH 8.0), 150 mM NaCl). A polyclonal anti-PepEST rabbit IgG was used at a 1:4000 dilution in 5% blocking solution. The anti- PepDef was used at a 1:3000 dilution. Proteins were detected using a 1:8000 dilution of mouse anti-rabbit IgG conjugated to peroxidase (Sigma) using ECL chemiluminescence blotting substrate (Amersham). The gel was stained with Coomassie Brilliant Blue.

ELISA

Proteins were isolated from leaf materials of transgenic plants and protein concentrations were determined in the crude extracts according to Bradford (1976). The soluble protein fractions were subjected to ELISA to determine the amount of PepEST or PepDef protein.

Resistance Evaluation of the Transgenic Plants

Spores of anthracnose fungus were cultured on a potato dextrose agar (PDA) medium at 28° C. The spores were collected and diluted in sterilized water. Then, the spore suspension was filtered through two layers of gauze, and the filtrate was centrifuged at 1,500 rpm for 5 min. The sediment composed of conidia was resuspended in sterilized water with the concentration adjusted to 5×10⁵/ml.

Inoculation with C. gloeosporioides was done by applying 10 μl of a spore suspension on mature unripe-green fruits. The fruits with drops of spore suspension were placed at high humidity for 2 days to stimulate infection by hyphal germination in dark at 28° C. Thereafter, the fruits were incubated further in a growth chamber. The infected fruits were collected separately from the drop-inoculated area. For the control, 10 μl of distilled water was applied on the unripe pepper fruits.

### EXAMPLES

### Example 1

### Construction of Plant Expression Vectors

A binary vector pCAMBIA1300 was used as a backbone for plant expression vectors. An expression cassette containing a resistance gene driven by CaMV35S promoter and Nos terminator was cloned into the multi-cloning site of pCAMBIA1300. The resulting expression vectors were carried PepEST and PepDef and named as pCAM-EST and pCAM-Def, respectively. The T-DNA region of the vector carries hygromycin phosphotransferase (HPT) gene driven by CaMV35S promoter and the gene expression cassette.

### Example 2

### Pepper Transformation

To optimize the regeneration condition for pepper explants, the explants were tested on various combinations of auxin and cytokinin; the best combination for shoot regeneration was IAA and Zeatin in 0.1-0.5 and 1-2 mg/L, respectively. Numerous genotypes tested were well regenerated under these conditions. The age of the plants had some influence on both regeneration and transformation. The aseptic plants should be germinated in dark for 7 days and then illuminated in light for 6 hours just before use. The explants should be isolated before the emergence of true leaf.

Agrobacterium tumefaciens was treated in a various manner, such as pH, temperature, chemicals, during the infection onto pepper explants. We then scored callus development on the infected explants under high dose of hygromycin B (20 mg/l). Callus formation efficiently occurred after inoculation on the medium at pH 5.5 at 26° C. The duration of incubation had effects on the transformation frequency. A longer incubation time resulted in browning of the pepper explants. Therefore, 2 days of incubation were optimal for cocultivation of the explants with Agrobacterium.

Since the explants were isolated from young hypocotyls and cotyledons, they retained efficient morphogenetic potentials for shoot development. Particularly, de novo regenerations occurred in the upper part of the explants. We can easily observe condensed axillary shoots from the green part of explants. Therefore, it is important to select the transformed cells under strict selection conditions because mild strength of antibiotics in the selection medium does not properly inhibit the growth of ‘false positive shoots’. Even more, the false positive shoots would completely block the division of transgenic cells. Greening of the explants can be inhibited by limiting light and with high concentrations of hygromycin B. Healthy transgenic callus developed from the cutting edge of the explants. Then, the transgenic callus was forced to regenerate shoots on the shoot induction medium containing 20 mg/l hygromycin B.

### Example 3

### Hygromycin Sensitivity of Pepper Cells

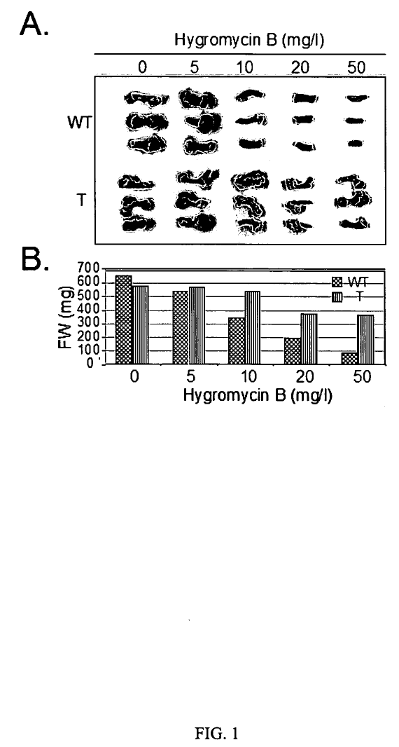

FIG. 1 shows the relative growth of pepper cells on SIM medium supplemented with 0, 5, 10, 20, and 100 mg/l hygromycin B. It can be seen that the antibiotic at a concentration of 5 mg/l showed a severe inhibitory effect on the growth after 14 days of culture. The transgenic cells showed resistance to hygromycin B up to 50 mg/l. Selection of transgenic cells was preformed in medium containing 20 mg/l hygromycin B in all experiments.

### Example 4

### Transgenic Lines Expressing GUS Gene

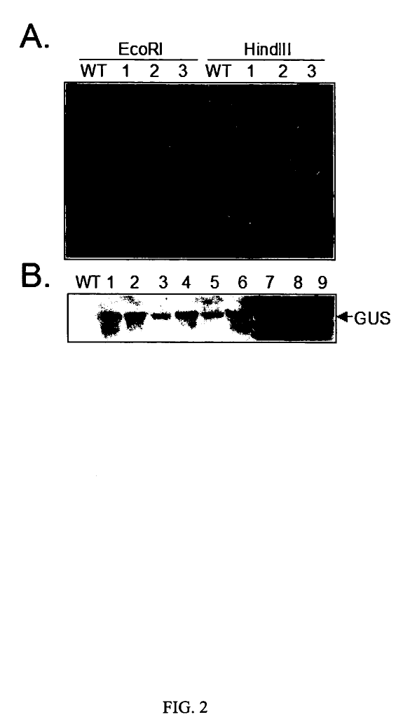

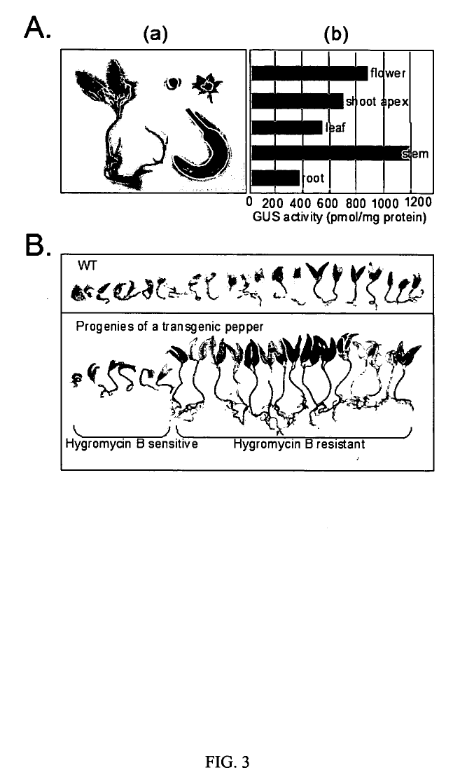

An efficient pepper transformation method was established on the basis of the shoot regeneration system of pepper on selection medium. To test the reliability of the transformation system, GUS reporter gene was introduced into pepper plants. The pepper explants inoculated with Agrobacterium carrying pCAMBIA1301 or 1304 was able to produce stably transformed callus and plants. The integration and expression of GUS gene were confirmed by Southern and Northern hybridizations, respectively (FIG. 2). The activity of GUS enzyme in transgenic peppers was assayed by histochemical and fluorometric methods. Inoculation of the explants with Agrobacterium carrying pCAMBIA 1301 was found to show no transient GUS activity. The lack of GUS activity was due to the presence of the CAT intron with the GUS coding frame of the pCAMBIA1301 vector. Results showed that expression of GUS gene under the control of CaMV35S promoter was consistent throughout the plant development in the transgenic peppers (FIG. 3A). All the progenies (T₁) of the transgenic plants showed an expected segregation pattern for hygromycin resistance, indicating that the T-DNA was stably maintained in the progeny through the generation (FIG. 3B).

### Example 5

### Transgenic Lines Over-Expressing PepEST Protein

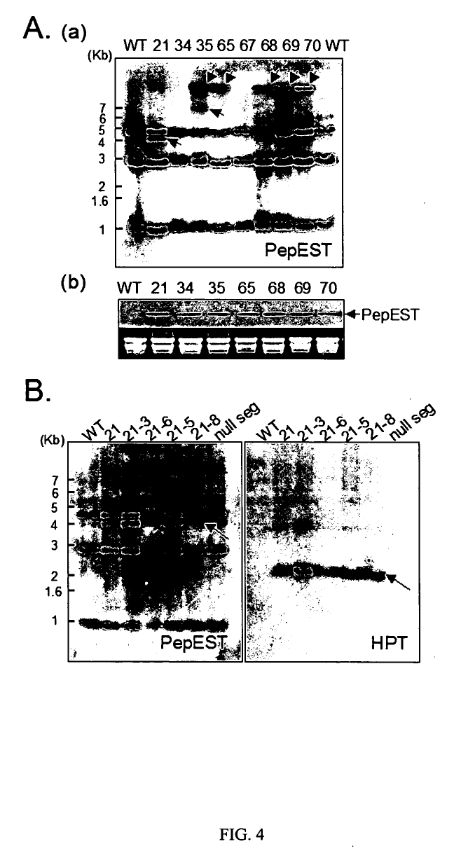

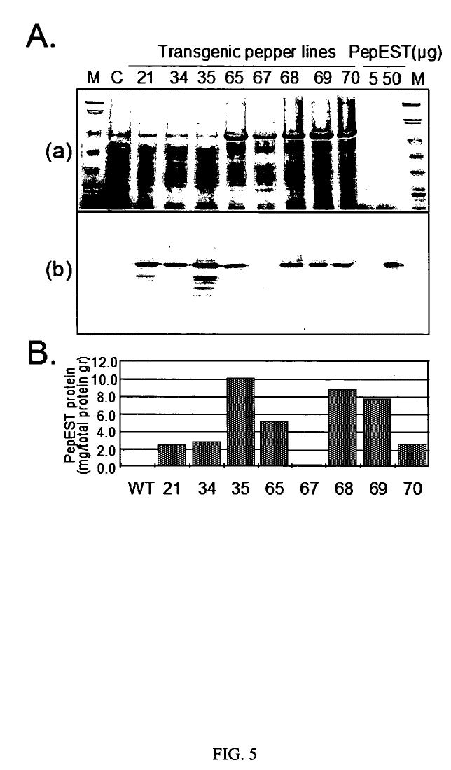

The PepEST gene, encoding an esterase, was cloned from the ripe pepper fruit that showed resistance to anthracnose fungus (Kim et al., 2001). To assess the function of the PepEST gene in disease resistance, its gene was introduced into pepper using Agrobacterium-mediated transformation. To express the 36.5 kD PepEST protein in the plant, the cDNA sequence was ligated into plasmid pCAMBIA1300 between the CaMV35S promoter and the Nos terminator. In the transgenic plants, Southern and Northern analyses were carried out to confirm the presence and expression of the transgene using PepEST sequences (FIG. 4A). Segregation analysis was also performed on the progenies by selecting the seedlings from selfed transgenic plants on media containing hygromycin B. The results indicate that the transgene was stably maintained through T-DNA integration into the genome of the plant (FIG. 4B). Accumulation of the PepEST protein was examined by Western blot and ELISA assays. A protein band specifically recognized by the PepEST polyclonal antiserum confirmed the expression of the PepEST protein in 7 transgenic plants (FIG. 5A). The PepEST accounted for 0.01% of the soluble proteins in the transgenic plants (FIG. 5B).

### Example 6

### Transgenic Lines Over-Expressing PepDef

The pepper defensin, PepDef, accumulated highly during fruit ripening. The role of PepDef was suggested to protect the reproductive organs against biotic and abiotic stresses (Oh et al., 1999). To generate transgenic resistant peppers against C. gloeosporioides based on the proposed function of PepDef, a chimeric construct was designed. Transcription of the PepDef gene was placed under the control of CaMV35S promoter and Nos terminator.

The construct was transformed into pepper using Agrobacterium strain GV3101. The regenerated plants displayed normal phenotypes compared with wild type peppers. To screen transgenic plants, PCR was conducted by the combination of a sequence from the CaMV35S promoter as a forward primer and a sequence from the 3′-untranslated region of PepDef cDNA as a reverse primer. Transgenic pepper seeds were collected from the individual transgenic lines (Data not shown).

### Example 7

### Disease Resistance against C. gloeosporioides in the Transgenic Pepper Fruits Expressing PepEST or PepDef

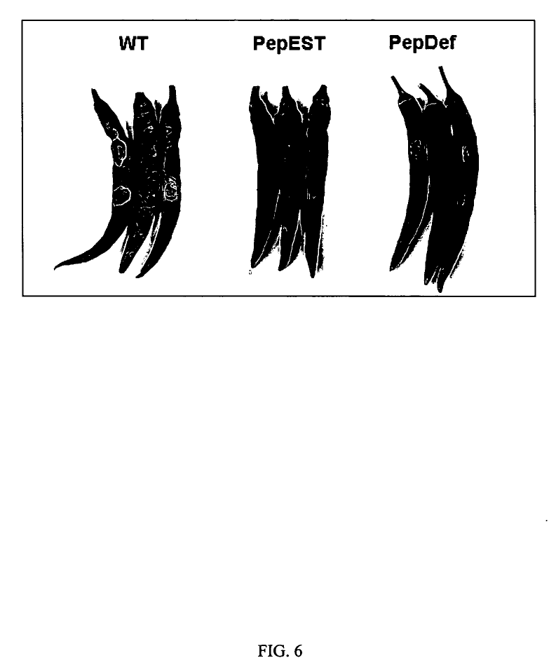

To assay the disease resistance in the transgenic plants, conidia of the virulent C. gloeosporioides were used to inoculate the unripe fruits of transgenic peppers. Transgenic fruits remained healthy but the unripe fruits from wild type plant developed typical anthracnose symptoms. As shown in FIG. 6, the transgenic fruits showed high levels of disease resistance against anthracnose. These results demonstrate the use of pepEST or PepDef as a novel source of genetic resistance to anthracnose in peppers. We further suggest that the transgenic peppers can be applied in the practical breeding to generate pepper lines resistant to the anthracnose fungus.

### REFERENCES

Jefferson R A, Kavanagh T A and Bevan M W (1987) GUS fusions: beta-glucuronidase as a sensitive and versatile gene fusion marker in higher plants. EMBO J 20: 3901-3907
Kim Y S, Lee H H, Ko Mk, Song C E, Bae C Y, Lee Y H and Oh B J (2001) Inhibition of fungal appressorium formation by pepper esterase. Molecular Plant-Microbe Interactions 14: 80-85

Oh B J, Ko M K, Kostenyuk I, Shin B C and Kim K S (1999) Coexpression of a defensin gene and a thionin-like gene via different signal transduction pathways in pepper and colletotrichum gloeosporioides interactions. Plant Molecular Biology 41: 313-319

1. An expression vector for transformation of pepper cells comprising:
a) a polynucleotide harboring a pepper defensin (PepDef) gene of SEQ ID NO:1 or pepper esterase (PepEST) gene of SEQ ID NO:2; and
b) regulatory sequences operatively linked to the polynucleotide such that the polynucleotide is expressed in the pepper cells,
wherein said expression vector transforms pepper explants comprising the pepper cells in MS medium supplemented with 0.5 mg/L of IAA and 0.2 mg/L of Zeatin under incubation.

2. A transgenic pepper cell transformed with the expression vector of claim 1, wherein said transgenic pepper cell is cultured in MS medium supplemented with 0.2 mg/L of IAA and 1 mg/L of Zeatin.

3. A transgenic pepper plant grown from the transgenic pepper cell of claim 2.

4. Progeny of the transgenic pepper plant of claim 3, wherein the progeny comprises the expression vector of claim 1.

5. A method of over-expressing pepper defensin (PepDef) or pepper esterase (PepEST) in a pepper plant, said method comprising:
i) integrating the vector of claim 1 into the genome of at least one cell of pepper explants by incubating said vector and said pepper explants in MS medium supplemented with 0.5 mg/L of IAA and 0.2 mg/L of Zeatin to produce at least one transformed pepper cell;
ii) culturing said at least one transformed pepper cell in MS medium supplemented with 0.2 mg/L of IAA and 1 mg/L of Zeatin to produce transgenic pepper cells; and
iii) growing said transgenic pepper cells to produce a transgenic pepper plant, wherein said polynucleotide encoding pepper defensin (PepDef) or pepper esterase (PepEST) is over-expressed in said transgenic pepper plant.

6-7. (canceled)


SEQUENCE LISTING (6 sequences)

SEQ ID NO: 1 - DNA, 225 bp, Capsicum annuum
atggctggcttttccaaagtggttgcaactatttttcttatgatgttgctggtttttgctactgatatgatggcggaggcaaagatctgcgaggcgttgagcggcaacttcaaggggttgtgccttagtagccgcgattgtggtaatgtttgccgtagagagggatttaccgatggctcttgcattggattccgtcttcaatgcttctgcacgaagccctgtgct

SEQ ID NO: 2 - DNA, 1217 bp, Capsicum annuum
ttatctgtgtgatcaattattatggctagccaaagttttgttcctccaatttttgaaaatccctttcttaacattgaagaattagcaggtgacacaattgtacgtaaacctgaacccctcacacaagccaattctgatcccaatggcacgtccttagttgtatctaaagacgtagaccttgacatcaacaaaaagacatggctgcgaatatacgtcccacaacgaataatcacaaatcataatgatgatgaaaaattgcctgtcattttctactaccatggtggaggctttgttttcttccatgccaatagttttgcctgggatttgttttgtcaaggacttgctggaaaccttggggcaatggttatctcccttgaatttcgtctggcccctgaaaatcgccttcctgcagcttacgacgatgccatggatgggttatattggattaaatcaactcaagatgaatgggtccgaaaatattcagatttgagtaacgtttatctttttggatctagttgcggtggaaacatagcttaccatgcagggttacgggtagcagctggggcatataaagaactagagccagtgaagatcaaagggctaattttgcatcaaccatatttcagtggaaaaaacaggacagaatctgaagagaagctaaaggatgatcaacttttgccattacatgcaattgacaaaatgttcgacttgtccttgccaaaagggacacttgatcatgatcatgaatattccaatccatttcttaatggagggtccaagcatttagatgatgtgatcgcacaaggctggaagattcttgtaactggtgtctctggagatcctctggttgataatgcgcgcaactttgcaaattttatggaagaaaaaggcataaaaactttcaagctctttggagatggttatcatgcaattgaggggtttgaaccatcaaaggcagcagctttaattggcgccaccaaagatttcatatgtgctactacaaattaaaaatatgtaacgtagcatcctgctagcgttgtgtttgtttcatttccttcaaataaatcaagtgagcttctttgtgcaaataagaggggtttacaccctccttcctgttagagattactttaaaatattatatttctcttgaagatcaaagttttagagatgagttattgctgaaaaaaaaaaaaaaaaaaaaaaaaaaaaaaaaaaaa

SEQ ID NO: 3 - DNA, 32 bp, Artificial Sequence
  primer
ggatccaaaatggctagccaaagttttgttcc

SEQ ID NO: 4 - DNA, 22 bp, Artificial Sequence
  primer
aatttgtagtagcacatatgaa

SEQ ID NO: 5 - DNA, 33 bp, Artificial Sequence
  primer
gggtctagaaaaatggctggcttttccaaagtg

SEQ ID NO: 6 - DNA, 30 bp, Artificial Sequence
  primer
ctcggatcctaattgagcacagggcttcgt